(19) United States
(12) Patent Application Publication
(10) Pub. No.: US 2016/0102285 A1
(43) Pub. Date: Apr. 14, 2016

(54) METHODS AND APPARATUS FOR CONTINUOUS ENZYMATIC HYDROLYSIS OF PRETREATED BIOMASS

(71) Applicant: API Intellectual Property Holdings, LLC, Atlanta, GA (US)

(72) Inventor: Vesa PYLKKANEN, Atlanta, GA (US)

(21) Appl. No.: 14/881,113

(22) Filed: Oct. 12, 2015

Related U.S. Application Data
(60) Provisional application No. 62/062,975, filed on Oct. 13, 2014.

(51) Int. Cl. C12M 1/00

(52) U.S. Cl. CPC C12M 47/06; C12M 23/58

(57) ABSTRACT

Some variations provide an apparatus for enzymatically hydrolyzing pretreated lignocellulosic biomass, including at least one clarifying vessel comprising: an inlet well, disposed with walls at or near the top of the clarifying vessel, configured for continuously feeding pretreated lignocellulosic biomass; overflow weirs to allow liquid-phase hydrolysate to be continuously recovered from the clarifying vessel; and a controllable bottom outlet to allow a solid phase to be continuously or periodically recovered from the clarifying vessel. In certain embodiments, the clarifying vessel is an existing recausticizing clarifier in a pulp and paper mill. Other variations provide a method of enzymatically hydrolyzing pretreated lignocellulosic biomass, comprising: introducing pretreated lignocellulosic biomass to a clarifying vessel in the presence of cellulase enzymes, to hydrolyze the cellulose to a liquid-phase hydrolysate; separating, by sedimentation, the hydrolysate from the lignin; and recovering the hydrolysate from overflow weirs of the clarifying vessel.

### PRIORITY DATA

This patent application is a non-provisional application claiming priority to U.S. Provisional Patent App. No. 62/062,975, filed Oct. 13, 2014, which is hereby incorporated by reference herein.

### FIELD OF THE INVENTION

The present invention generally relates to methods and systems for preparing fermentable sugars from lignocellulosic biomass.

### BACKGROUND OF THE INVENTION

Enzymatic hydrolysis is a key process for a biorefinery based on production of sugars. The rate of enzymatic hydrolysis, final carbohydrate conversion, and concentration all critically affect the technoeconomic feasibility of commercial operations. Enzymatic hydrolysis performed at high solids loading offers several advantages over low solids loading, because of higher sugar and bioproduct concentrations, smaller equipment, less energy for heating and cooling of the slurry, and lower hydraulic loads. Therefore, enzymatic hydrolysis at high solids loadings is highly desirable to develop an economically viable process. See Geng et al., “Strategies to achieve high-solids enzymatic hydrolysis of dilute-acid pretreated corn stover,” Bioresource Technology 187 (2015) 43-48.

However, hydrolysis at high insoluble solids introduces a lack of available water in the reactor. Water is essential to the hydrolysis and conversion of lignocellulosic biomass since it is the key medium for enzymes to diffuse in and for products to diffuse away from reaction sites. Water also reduces the viscosity of the slurry by increasing the lubricity of the particles, which decreases the required shear stress necessary to produce a given shear rate, allowing lower power input for mixing during hydrolysis. Therefore, high-solids hydrolysis can create rheological challenges, cause insufficient mixing, reduce mass- and heat-transfer efficiency, and increase the concentration of enzymes inhibitors in the system, resulting in low conversion of carbohydrates into fermentable sugars.

To overcome the challenges of enzymatic hydrolysis at high solids and make the overall conversion process more economically viable, several approaches have been developed, including fed-batch, splitting/thickening, and clarifier processes.

For the fed-batch process, substrates and/or enzymes are introduced into a hydrolysis reactor successively. The fed-batch system allows time for the slurry to liquefy before adding additional solids and a low initial insoluble solids content can be kept. In order to maintain high rates of carbohydrate conversion of hydrolysis, it is important to find an optimal point to add solids into the system, which is highly dependent on substrate characteristics and enzyme dosage/type.

For the splitting/thickening process, pretreated substrate is mixed with part of the enzymes at a lower solids loading and then filtered to obtain the desired solids loading. Using split addition, the solids content can be increased to 20% while maintaining the enzymatic hydrolysis conversion efficiency comparable to that with 5% total solids.

In known clarifier processes, a gravity clarifier separates the partially hydrolyzed stream into a sugar stream and unhydrolyzed solids, and the sugar stream is used to dilute the initial insoluble solids content. This process can reduce the initial viscosity without decreasing the final sugar concentration because of the high sugar concentration of the sugar stream.

Improvements are still desired to enable a continuous process and to reach high solids concentrations in enzymatic hydrolysis of pretreated biomass.

### SUMMARY OF THE INVENTION

The present invention addresses the aforementioned needs in the art.

Some variations provide an apparatus for enzymatically hydrolyzing pretreated lignocellulosic biomass, the apparatus including at least one clarifying vessel comprising:

(a) an inlet well, disposed with walls at or near the top of the clarifying vessel, configured for continuously feeding pretreated lignocellulosic biomass;

(b) one or more overflow weirs configured to allow liquid-phase hydrolysate to be continuously or periodically recovered from the clarifying vessel; and

(c) a controllable bottom outlet to allow a solid phase or solid slurry to be continuously or periodically recovered from the clarifying vessel, separately from the liquid-phase hydrolysate.

In some embodiments, the walls of the well extend down below the local elevation (i.e., height) of the weirs.

In some embodiments, the controllable bottom outlet is further configured with a rake for continuously or periodically assisting in removal of the solid phase or solid slurry.

The clarifying vessel is preferably capable of operating at a solids concentration of at least 10 wt %, or at least 20 wt %.

The apparatus may further comprise a recycle line and pump for recycling at least a portion of the hydrolysate back to the inlet well.

The apparatus may include at least two clarifying vessels in series, in parallel, or when there are three or more clarifying vessels, both series and parallel configurations.

In certain embodiments, the clarifying vessel is an existing recausticizing clarifier in a pulp and paper mill. When the apparatus includes at least two clarifying vessels in series or in parallel, each of the clarifying vessels may be an existing recausticizing clarifier in a pulp and paper mill.

Other variations provide a method of enzymatically hydrolyzing pretreated lignocellulosic biomass, the method comprising:

(a) providing pretreated lignocellulosic biomass containing cellulose and lignin;

(b) introducing the pretreated lignocellulosic biomass to a clarifying vessel under effective hydrolysis conditions and in the presence of enzymes including cellulases, to hydrolyze the cellulose to a liquid-phase hydrolysate;

(c) separating, by sedimentation within the clarifying vessel, the hydrolysate from the lignin; and

(d) recovering the hydrolysate from one or more overflow weirs of the clarifying vessel.

In various embodiments, the pretreated lignocellulosic biomass is chemically pretreated, physically pretreated, or a combination thereof and is optionally premixed with enzymes. The pretreated lignocellulosic biomass is preferably introduced into a well, disposed with walls, at or near the top of the clarifying vessel.

The effective hydrolysis conditions may include a hydrolysis residence time selected from about 4 hours to about 72 hours; and/or a hydrolysis pH selected from about 4 to about 7; and/or a hydrolysis temperature selected from about 30° C. to about 70° C. The method may be continuous, semi-continuous, batch, or fed-batch.

The lignin may be recovered continuously or periodically from a bottom stream output (optionally with a rake for assisting in solid removal) of the clarifying vessel.

In some embodiments, the method further comprises introducing a flocculating agent to the clarifying vessel. In these or other embodiments, the method further comprises introducing a surfactant to the clarifying vessel. In certain embodiments, a lignin-derived compound, different from the lignin fed with the pretreated lignocellulosic biomass, is introduced and has surfactant properties, flocculating properties, or both surfactant and flocculating properties.

Step (b) is preferably conducted at a solids concentration of at least 10 wt %, at least 15 wt %, at least 20 wt %, or more.

### BRIEF DESCRIPTION OF THE FIGURES

FIG. 1 is a simplified block-flow diagram depicting an apparatus of some embodiments of the present invention.

FIG. 2 is a simplified block-flow diagram depicting a method of some embodiments of the present invention.

### DETAILED DESCRIPTION OF EMBODIMENTS OF THE INVENTION

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying drawings.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. All composition numbers and ranges based on percentages are weight percentages, unless indicated otherwise. All ranges of numbers or conditions are meant to encompass any specific value contained within the range, rounded to any suitable decimal point.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of.”

Some variations of the present invention provide methods and apparatus for continuous enzymatic saccharification of cellulosic material with separation of the hydrolyzed cellulose from non-cellulosic material and/or non-hydrolyzed material.

In some embodiments, a pulp suspension is added continuously or semi-continuously to a clarifying vessel, wherein clarified cellulose monomers are raised to the top, while heavier particles (lignin, ash, etc.) are drawn from the bottom.

A “clarifying vessel” or “clarifier” generally is a settling tank, container, or vessel with mechanical means for continuous or periodic removal of solids being deposited by sedimentation. A clarifier is generally used to remove solid particulates or suspended solids from a liquid for clarification and/or thickening.

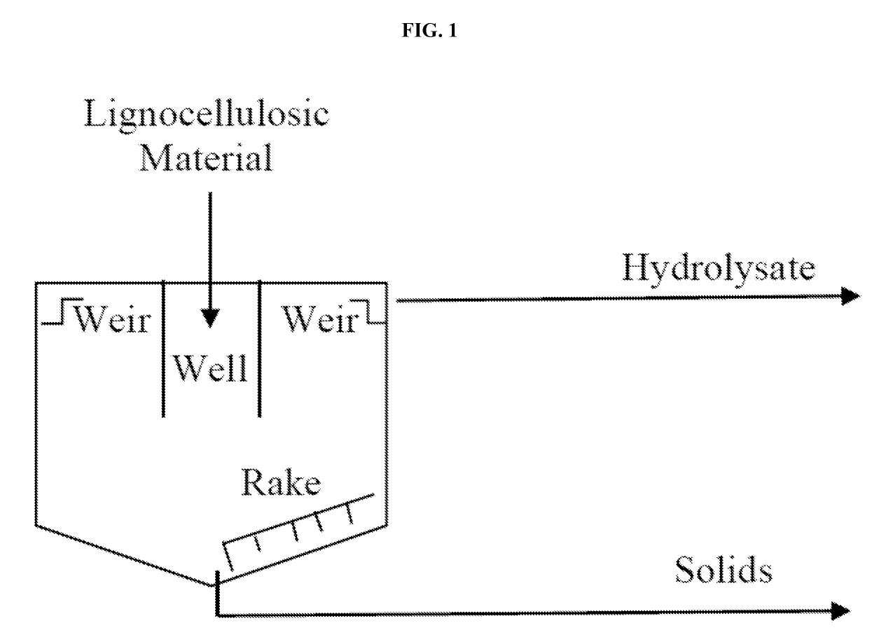

Reference is made to an exemplary apparatus in FIG. 1. In some embodiments herein, a lignocellulosic material is introduced in the top-center of a large vessel. The introduction of lignocellulosic material may be in a well with walls, as depicted in FIG. 1, to avoid mixing near the top of the vessel and short-circuit of feed material out of the clarifier before separation can occur. The feed material may be premixed with enzymes, including cellulases and optionally hemicellulases. Alternatively or additionally, enzymes may be added directly to the clarifying vessel.

The residence time in the clarifying vessel is selected to be sufficient to cause cellulose liquefaction, which is at least partial cellulose hydrolysis resulting in soluble cellulose oligomers or monomers (referred to herein as “liquefied cellulose”). For example, the residence time may be about 4, 8, 12, 24, 36, 72 hours or more. With sufficient time and enzymes, the liquefied cellulose will break down further into monomeric sugars, such as glucose, xylose, mannose, galactose, and arabinose. The clarifying vessel is eventually filled with liquefied cellulose and solids, wherein the solids include lignin and potentially cellulose that is not liquefied.

The driving force for the clarifying separation is the difference in density between the solid and the liquid. Ordinarily, sedimentation is effected by the force of gravity. The density of cellulose and sugar components is about 1.5 g/cm³ (dry basis), while the density of lignin is about 1.3 g/cm³ and the density of ash is about 2.7 g/cm³. The sugars and oligomers are diluted in water within the liquefied cellulose, so that the density of the hydrolysate is closer to 1.0 g/cm³. Therefore, the solid particles will tend to settle to the bottom of a solution of liquefied cellulose (hydrolysate). When the clarifying vessel is full or nearly full, the lower-density hydrolysate will overflow from the edges, commonly called weirs (see FIG. 1). Meanwhile, the bottom solids are continuously or periodically removed from the bottom-center to maintain the solids level. The removal may be assisted by a rake, which provides a gentle mixing.

For a given density difference, the solid settling process proceeds more rapidly for larger-sized particles. Particles too minute to settle at practical rates may form flocs by the addition of flocculating agents such as sodium silicate, alum, lime, or alumina. The agglomerated particles act like a single large particle, improving settling rate.

The pH in the clarifier may be selected from about 4 to about 7, such as from about 4.5 to about 6.5, for example. The pH of the clarifier contents may be periodically adjusted using acid or base to maintain optimum enzyme performance. The temperature may also adjusted by direct or indirect heating means, to a hydrolysis temperature of about 30° C., 35° C., 40° C., 50° C., 60° C., 70° C. or more, for example. Surfactants and other additives may be introduced, to assist cellulose hydrolysis.

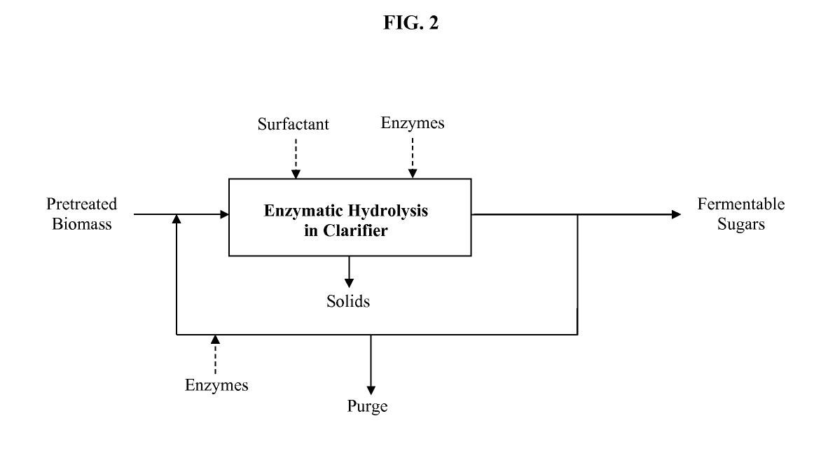

There may be several clarifying vessels in series or parallel to accommodate maximum saccharification sugar yield. The consistency (solids concentration) of the cellulosic material within the clarifier should be at least 5 wt %, preferably at least 10 wt % and more preferably at least 20 wt %, to obtain maximum concentration of the hydrolyzed sugars. The mixing of enzymes at high cellulose consistency becomes increasingly difficult as the solids concentration increases. By continuously liquefying part of the cellulosic material, the viscosity is reduced to allow efficient mass transfer of the enzymes. Also, removal of the hydrolysate from the top of the clarifier provides a relatively pure cellulosic sugar steam at high concentration. A portion of the hydrolysate may be recycled, optionally with a purge and input of additional enzymes, as shown in FIG. 2.

In some embodiments, one or more existing recausticizing clarifiers in a pulp and paper mill are reused as clarifying vessels capable of carrying out enzymatic hydrolysis.

Certain exemplary embodiments of the invention will now be described. These embodiments are not intended to limit the scope of the invention as claimed. The order of steps may be varied, some steps may be omitted, and/or other steps may be added. Reference herein to first step, second step, etc. is for illustration purposes only.

Some variations provide an apparatus for enzymatically hydrolyzing pretreated lignocellulosic biomass, the apparatus including at least one clarifying vessel comprising:

(a) an inlet well, disposed with walls at or near the top of the clarifying vessel, configured for continuously feeding pretreated lignocellulosic biomass;

(b) one or more overflow weirs configured to allow liquid-phase hydrolysate to be continuously or periodically recovered from the clarifying vessel; and

(c) a controllable bottom outlet to allow a solid phase or solid slurry to be continuously or periodically recovered from the clarifying vessel, separately from the liquid-phase hydrolysate.

In some embodiments, the walls of the well extend down below the local elevation (i.e., height) of the weirs.

In some embodiments, the controllable bottom outlet is further configured with a rake for continuously or periodically assisting in removal of the solid phase or solid slurry.

The clarifying vessel is preferably capable of operating at a solids concentration of at least 10 wt %, or at least 20 wt %.

The apparatus may further comprise a recycle line and pump for recycling at least a portion of the hydrolysate back to the inlet well.

The apparatus may include at least two clarifying vessels in series, in parallel, or when there are three or more clarifying vessels, both series and parallel configurations.

In certain embodiments, the clarifying vessel is an existing recausticizing clarifier in a pulp and paper mill. When the apparatus includes at least two clarifying vessels in series or in parallel, each of the clarifying vessels may be an existing recausticizing clarifier in a pulp and paper mill.

Other variations provide a method of enzymatically hydrolyzing pretreated lignocellulosic biomass, the method comprising:

(a) providing pretreated lignocellulosic biomass containing cellulose and lignin;

(b) introducing the pretreated lignocellulosic biomass to a clarifying vessel under effective hydrolysis conditions and in the presence of enzymes including cellulases, to hydrolyze the cellulose to a liquid-phase hydrolysate;

(c) separating, by sedimentation within the clarifying vessel, the hydrolysate from the lignin; and

(d) recovering the hydrolysate from one or more overflow weirs of the clarifying vessel.

In various embodiments, the pretreated lignocellulosic biomass is chemically pretreated, physically pretreated, or a combination thereof and is optionally premixed with enzymes. The pretreated lignocellulosic biomass is preferably introduced into a well, disposed with walls, at or near the top of the clarifying vessel.

The effective hydrolysis conditions may include a hydrolysis residence time selected from about 4 hours to about 72 hours; and/or a hydrolysis pH selected from about 4 to about 7; and/or a hydrolysis temperature selected from about 30° C. to about 70° C. The method may be continuous, semi-continuous, batch, or fed-batch.

The lignin may be recovered continuously or periodically from a bottom stream output (optionally with a rake for assisting in solid removal) of the clarifying vessel.

In some embodiments, the method further comprises introducing a flocculating agent to the clarifying vessel. In these or other embodiments, the method further comprises introducing a surfactant to the clarifying vessel. In certain embodiments, a lignin-derived compound, different from the lignin fed with the pretreated lignocellulosic biomass, is introduced and has surfactant properties, flocculating properties, or both surfactant and flocculating properties. The lignin may be hardwood AVAP® lignin, for example.

Step (b) is preferably conducted at a solids concentration of at least 10 wt %, at least 15 wt %, at least 20 wt %, or more.

In some embodiments, the method further comprises an oligomer hydrolysis step comprising heat treatment and/or acid treatment of the glucose oligomers to generate additional glucose monomer. Optionally, at least a portion of the glucose oligomers may be removed from a circulation line to reduce glucose oligomer inhibition of cellulose hydrolysis. The removal of glucose oligomers may target cellobiose, which is a relatively high enzyme inhibition effect, compared to larger oligomers.

In various embodiments, the pretreated lignocellulosic biomass is chemically pretreated (e.g., with an acid or base), physically pretreated (e.g., refined or exploded), or a combination thereof. The pretreated lignocellulosic biomass may be a pulp material, derived from wood or lignocellulosic biomass, selected from the group consisting of kraft pulp, sulfite pulp, soda pulp, mechanical pulp, thermomechanical pulp, chemimechanical pulp, and combinations thereof.

In certain embodiments, the pretreated lignocellulosic biomass is GP3+™ pulp derived from wood or lignocellulosic biomass. The pretreated lignocellulosic biomass may be obtained from steam or hot-water extraction of lignocellulosic biomass, optionally followed by evaporation to remove water and potentially fermentation inhibitors (e.g., acetic acid).

In certain embodiments, the pretreated lignocellulosic biomass is AVAP® pulp derived from wood or lignocellulosic biomass. The pretreated lignocellulosic biomass may be obtained from fractionation of lignocellulosic biomass in the presence of water, an acid catalyst, and a solvent for lignin, optionally followed by evaporation to remove water and potentially fermentation inhibitors (e.g., acetic acid).

In preferred embodiments, the pretreated lignocellulosic biomass is present in the hydrolysis reactor (i.e., the clarifying vessel) at a solids concentration of about 15 wt % or more, about 20 wt % or more, about 25 wt % or more, or about 30 wt % or more.

Enzymes may be introduced to the pretreated lignocellulosic biomass at multiple times and/or locations. The concept of split addition of enzymes may be applied. In some embodiments, a first amount of enzymes is introduced at a first solids concentration, a second amount of enzymes is introduced at a second solids concentration, the second solids concentration being higher than the first solids concentration.

Also provided is a system configured for carrying out a method as described.

Also provided is a sugar product produced by a process comprising a method as described. A fermentation product (e.g., ethanol) may be derived from the sugar product.

In some variations, biomass may be subjected to a steam or hot-water soak to dissolved hemicelluloses, with or without acetic acid recycle. This step is followed by mechanical refining, such as in a hot-blow refiner, of the cellulose-rich (and lignin-rich) solids. The refined solids are then enzymatically hydrolyzed to generate sugars. A stripping and/or evaporating step for removing fermentation inhibitors in the hydrolysate may be included.

In some embodiments, the reaction solution comprises steam in saturated, superheated, or supersaturated form. In some embodiments, the reaction solution comprises pressurized hot water.

In some embodiments, the reaction solution further comprises an acid, such as a sulfur-containing acid (e.g., sulfuric acid, sulfurous acid, or sulfur dioxide), acetic acid, formic acid, or others. The acid may include acetic acid recovered from the digested stream.

The mechanical refiner may be selected from the group consisting of a hot-blow refiner, a hot-stock refiner, a blow-line refiner, a disk refiner, a conical refiner, a cylindrical refiner, an in-line defibrator, a homogenizer, and combinations thereof. In some embodiments, the mechanical refiner is a blow-line refiner. Other mechanical refiners may be employed, and chemical refining aids may be introduced.

Mechanically treating (refining) may employ one or more known techniques such as, but by no means limited to, milling, grinding, beating, sonicating, or any other means to reduce cellulose particle size. Such refiners are well-known in the industry and include, without limitation, Valley beaters, single disk refiners, double disk refiners, conical refiners, including both wide angle and narrow angle, cylindrical refiners, homogenizers, microfluidizers, and other similar milling or grinding apparatus. See, for example, Smook, Handbook for Pulp & Paper Technologists, Tappi Press, 1992.

A blow tank may be situated downstream of the mechanical refiner, so that the mechanical refiner operates under pressure. The pressure of the mechanical refiner may be the same as the digestor pressure, or it may be different. In some embodiments, the mechanical refiner is operated at a refining pressure selected from about 30 psig to about 300 psig, such as about 40, 50, 60, 70, 80, 90, 100, 110, 120, 130, 140, 150, 160, 170, 180, 190, 200, 210, 220, 230, 240, 250, 260, 270, 280, or 290 psig.

A blow tank may be situated upstream of the mechanical refiner, so that the mechanical refiner operates under reduced pressure or atmospheric pressure. In some embodiments, the mechanical refiner is operated a refining pressure of less than about 50, 45, 40, 35, 30, 25, 20, 15, 10, or 5 psig, or at or about atmospheric pressure (0 psig).

In certain embodiments of the invention, a first blow tank is situated upstream of the mechanical refiner and a second blow tank is situated downstream of the mechanical refiner. In this scenario, the pressure is reduced somewhat between the digestor and the refiner, which operates above atmospheric pressure. Following the refining, the pressure is released in the second blow tank. In some embodiments, the mechanical refiner is operated at a refining pressure selected from about 10 psig to about 150 psig, such as about 20 psig to about 100 psig, or about 25, 30, 35, 40, 45, 50, 55, 60, 65, 70, or 75 psig.

A pressurized refiner may operate at the same pressure as the digestor, or at a different pressure. In some embodiments, both the digestor and the refiner operate in a pressure range corresponding to equilibrium steam saturation temperatures from about 170° C. to about 210° C., such as about 180° C. to about 200° C. In some embodiments, a pressurized refiner is fed by a screw between the digestor and the refiner.

In principle, the pressure in the refiner could be higher than the digestor pressure, due to mechanical energy input. For example, a high-pressure screw feeder could be utilized to increase pressure, if desired, in refining. Also, it will be recognized that localized pressures (forces) may be higher than the vapor pressure, due to the presence of mechanical surface force (e.g., plates) impacting the solid material or slurry.

In some embodiments, the vapor is separated from a blow tank, and heat is recovered from at least some of the vapor. At least some of the vapor may be compressed and returned to the digestor. Some of the vapor may be purged from the process.

The enzymes introduced or present in the enzymatic hydrolysis unit may include cellulases and optionally hemicellulases. The enzymes may include endoglucanases and exoglucanases.

The process may further include removal of one or more fermentation inhibitors by stripping.

The process may further include a step of fermenting the fermentable sugars to a fermentation product; and concentrating and purifying the fermentation product. In various embodiments, the fermentation product may be selected from ethanol, n-butanol, 1,4-butanediol, succinic acid, lactic acid, or combinations thereof.

Some embodiments further include comprising removing a solid stream containing lignin prior to fermentation of the fermentable sugars. In these or other embodiments, the process may further include removing a solid stream containing lignin following fermentation of the fermentable sugars. The lignin may be combusted or use for other purposes.

In some embodiments, enzymes are introduced directly to the mechanical refiner. In these or other embodiments, the enzymes are introduced to the digested stream, upstream of the mechanical refiner. The enzymes may include cellulases (e.g., endoglucanases and exoglucanases) and hemicellulases.

The effective hydrolysis conditions may include a maximum temperature of 75° C. or less, preferably 65° C. or less, within the mechanical refiner. In some embodiments, the effective hydrolysis conditions include a hydrolysis temperature of about 30, 35, 40, 45, 50, 55, 60, 65, or 70° C. within the mechanical refiner. These are average temperatures within the refining zone. Local hot spots may be present within the refiner, such as in regions of high-shear contact between cellulose-rich solids and metal plates.

The reaction solution may comprise hot water or steam in saturated, superheated, or supersaturated form. In some embodiments, the reaction solution further comprises an acid, such as a sulfur-containing acid. In some embodiments, the reaction solution further comprises acetic acid, which may be (at least in part) acetic acid recovered from the digested stream.

The mechanical refiner may be selected from the group consisting of a hot-blow refiner, a hot-stock refiner, a blow-line refiner, a disk refiner, a conical refiner, a cylindrical refiner, an in-line defibrator, a homogenizer, and combinations thereof. In certain embodiments, the mechanical refiner is one or more blow-line refiners.

In some embodiments, a blow tank is situated upstream of the mechanical refiner. The mechanical refiner is preferably operated at or about atmospheric pressure, due to the presence of enzymes.

In some embodiments, vapor is separated from a blow tank, and heat is recovered from at least some of the vapor. Some or all of the vapor may be compressed and returned to the digestor. Some of the vapor may be purged from the process.

The process may also include removal of one or more fermentation inhibitors by stripping. The stripping may be conducted prior to fermentation. One fermentation inhibitor is acetic acid, which may be recycled to the digestor.

The process may further include a step of fermenting the fermentable sugars to a fermentation product, such as ethanol, n-butanol, 1,4-butanediol, succinic acid, lactic acid, or combinations thereof. The fermentation product may be concentrated and purified.

The process may further include recovering a solid stream containing prior to fermentation of the fermentable sugars and/or following fermentation of the fermentable sugars. The lignin may be recovered for various uses, such as combustion (energy).

In some embodiments, a blow tank is situated downstream of the mechanical refiner. In other embodiments, a blow tank is situated upstream of the mechanical refiner. In certain embodiments, a first blow tank is situated downstream of the mechanical refiner and a second blow tank is situated upstream of the mechanical refiner.

Note that “blow tank” should be broadly construed to include not only a tank but any other apparatus or equipment capable of allowing a pressure reduction in the process stream. Thus a blow tank may be a tank, vessel, section of pipe, valve, separation device, or other unit.

In some embodiments, following a digestor to remove hemicellulose, an intermediate blow is performed to, for example, about 40 psig. The material is sent to a blowline refiner, and then to a final blow to atmospheric pressure.

In some embodiments, a cold blow discharger is utilized to feed a pressurized refiner. In some embodiments, a transfer conveyor is utilized to feed a pressurized refiner.

The refining may be conducted at a wide range of solids concentrations (consistency), including from about 2% to about 50% consistency, such as about 4%, 6%, 8%, 10%, 15%, 20%, 30%, 35%, or 40% consistency.

In some embodiments, heat is recovered from at least some of the vapor, using the principles of heat integration. At least some of the vapor may be compressed and returned to the digestor. Some of the vapor may be purged from the process.

In some embodiments, enzymes introduced or present in the enzymatic hydrolysis unit may include not only cellulases but also hemicellulases. In certain embodiments, enzymes introduced or present in the enzymatic hydrolysis unit include endoglucanases and exoglucanases.

The reaction solution optionally includes an acid catalyst, to assist in extraction of hemicelluloses from the starting material, and possibly to catalyze some hydrolysis. In some embodiments, the acid is a sulfur-containing acid (e.g., sulfur dioxide). In some embodiments, the acid is acetic acid, which may be recovered from the digested stream (i.e., from downstream operations).

The starting feedstock may include sucrose, such as in the case of energy cane. A majority of the sucrose may be recovered as part of the fermentable sugars.

The process may include cleaning the starting feedstock, by wet or dry cleaning. The process may include size reduction, hot-water soaking, dewatering, steaming, or other operations, upstream of the digestor.

The process may further include removal of one or more fermentation inhibitors (such as acetic acid or furfural) by stripping. This stripping may be conducted following step (e), i.e. treating the hydrolyzed cellulose stream, prior to fermentation. Alternatively, or additionally, the stripping may be conducted on a stream following digestion, such as in the blow line, or as part of an acetic acid recycle system.

The process may further include a step of fermenting the fermentable sugars to a fermentation product. Typically the process will further include concentration and purification of the fermentation product. The fermentation product may be selected from ethanol, n-butanol, 1,4-butanediol, succinic acid, lactic acid, or combinations thereof, for example. The lignin may be combusted for energy production, for example.

In some embodiments, the extraction solution comprises steam in saturated, superheated, or supersaturated form. In some embodiments, the extraction solution comprises hot water. Additives may be present, such as acid or base catalysts, or other compounds present in recycled streams. The fraction of starting hemicellulose that is extracted into solution may be from about 60% to about 95%, such as about 75%, 80%, 85%, or 90%.

In some embodiments, the process includes washing the cellulose-rich solids using an aqueous wash solution, to produce a wash filtrate; and optionally combining at least some of the wash filtrate with the extract liquor. In some of these embodiments, the process further includes pressing the cellulose-rich solids to produce the washed cellulose-rich solids and a press filtrate; and optionally combining at least some of the press filtrate with the extract liquor.

The process may include countercurrent washing, such as in two, three, four, or more washing stages. The separation/washing may be combined with the application of enzymes, in various ways.

In some embodiments, a unit is configured to cause at least some liquefaction as a result of enzymatic action on the cellulose-rich solids. “Liquefaction” means partial hydrolysis of cellulose to form glucose oligomers (i.e. glucan) that dissolve into solution, but not total hydrolysis of cellulose to glucose monomers (saccharification). Various fractions of cellulose may be hydrolyzed during liquefaction. In some embodiments, the fraction of cellulose hydrolyzed may be from about 5% to about 90%, such as about 10% to about 75% (e.g. about 15%, 20%, 25%, 30%, 35%, 40%, 45%, 50%, 55%, 60%, 65%, or 70%). In certain embodiments, there is no separate liquefaction tank or reactor; liquefaction and hydrolysis occur in the same vessel (e.g., refiner or hydrolysis reactor).

Some embodiments utilize a liquefaction-focused blend of enzymes in the clarifying vessel. A “liquefaction-focused blend of enzymes” means a mixture of enzymes that includes at least one enzyme capable of hydrolyzing cellulose to form soluble oligomers. In some embodiments, a liquefaction-focused blend of enzymes includes both endoglucanases and exoglucanases. Endoglucanases are cellulases that attack low-crystallinity regions in the cellulose fibers by endoaction, creating free chain-ends. Exoglucanases or cellobiohydrolases are cellulases that hydrolyze the 1,4-glycocidyl linkages in cellobiose.

Various cellulase enzymes may be utilized in the liquefaction-focused blend of enzymes, such as one or more enzymes recited in Verardi et al., “Hydrolysis of Lignocellulosic Biomass: Current Status of Processes and Technologies and Future Perspectives,” Bioethanol, Prof. Marco Aurelio Pinheiro Lima (Ed.), ISBN: 978-953-51-0008-9, InTech (2012), which is hereby incorporated by reference.

Some embodiments employ thermotolerant enzymes obtained from thermophilic microrganisms. The thermophilic microrganisms can be grouped in thermophiles (growth up to 60° C.), extreme thermophiles (65-80° C.) and hyperthermophiles (85-110° C.). The unique stability of the enzymes produced by these microrganisms at elevated temperatures, extreme pH and high pressure (up to 1000 bar) makes them valuable for processes at harsh conditions. Also, thermophilic enzymes have an increased resistance to many denaturing conditions such as the use of detergents which can be an efficient means to obviate the irreversible adsorption of cellulases on the substrates. Furthermore, the utilization of high operation temperatures, which cause a decrease in viscosity and an increase in the diffusion coefficients of substrates, have a significant influence on the cellulose solubilization. It is worth noting that most thermophilic cellulases do not show inhibition at high level of reaction products (e.g. cellobiose and glucose). As consequence, higher reaction rates and higher process yields are expected. The high process temperature also reduces contamination. See Table 6, “Thermostable cellulases” in Verardi et al., cited previously, for exemplary thermotolerant enzymes that may be used in the liquefaction-focused blend of enzymes.

In some embodiments, an enzyme is selected such that at a high temperature, the enzyme is able to catalyze liquefaction (partial hydrolysis) but not saccharification (total hydrolysis). When the temperature is reduced, the same enzyme is able to catalyze saccharification to produce glucose.

When the hydrolysis process employs enzymes, these enzymes will typically contain cellulases and hemicellulases. The cellulases here may include β-glucosidases that convert cellooligosaccharides and disaccharide cellobiose into glucose. There are a number of enzymes that can attack hemicelluloses, such as glucoronide, acetylesterase, xylanase, β-xylosidase, galactomannase and glucomannase. Exemplary acid catalysts include sulfuric acid, sulfur dioxide, hydrochloric acid, phosphoric acid, and nitric acid.

In some embodiments, non-acid and non-enzyme catalysts may be employed for co-hydrolyzing the glucose oligomers and the hemicellulose oligomers. For example, base catalysts, solid catalysts, ionic liquids, or other effective materials may be employed.

The process further comprises a step of fermenting the fermentable sugars to a fermentation product (such as ethanol), in some embodiments.

In some embodiments, the first hydrolysis catalyst includes cellulases. In some embodiments, the second hydrolysis catalyst includes hemicellulases. In other embodiments, the first hydrolysis catalyst and the second hydrolysis catalyst are acid catalysts, base catalysts, ionic liquids, solid catalysts, or other effective materials. The first hydrolysis catalyst may be the same as, or different than, the second hydrolysis catalyst.

In some embodiments, the glucose is recovered in a separate stream from the hemicellulose monomers. In other embodiments, the glucose and the hemicellulose monomers are recovered in the same stream. The process may include fermentation of the glucose and/or the fermentable hemicellulose sugars to a fermentation product.

The biomass feedstock may be selected from hardwoods, softwoods, forest residues, agricultural residues (such as sugarcane bagasse), industrial wastes, consumer wastes, or combinations thereof. In any of these processes, the feedstock may include sucrose. In some embodiments with sucrose present in the feedstock, a majority of the sucrose is recovered as part of the fermentable sugars. In order to preserve sucrose (when present), it is preferred to utilize enzymes rather than acid catalysts for cellulose hydrolysis.

In some embodiments, the process starts as biomass is received or reduced to approximately ¼″ thickness. In a first step of the process, the biomass is fed (e.g., from a bin) to a pressurized extraction vessel operating continuously or in batch mode. The biomass may first be steamed or water-washed to remove dirt and entrained air. The biomass may be immersed with aqueous liquor or saturated vapor and heated to a temperature between about 100° C. to about 250° C., for example 150° C., 160° C., 170° C., 180° C., 190° C., 200° C., or 210° C. Preferably, the biomass is heated to about 180° C. to 210° C.

The pressure in the pressurized vessel may be adjusted to maintain the aqueous liquor as a liquid, a vapor, or a combination thereof. Exemplary pressures are about 1 atm to about 30 atm, such as about 3 atm, 5 atm, 10 atm, or 15 atm.

The solid-phase residence time for the digestor (pressurized extraction vessel) may vary from about 2 minutes to about 4 hours, such as about 5 minutes to about 1 hour. In certain embodiments, the digestor residence time is controlled to be about 5 to 15 minutes, such as 5, 6, 7, 8, 9, 10, 11, 12, 13, 14 or 15 minutes. The liquid-phase residence time for the digestor may vary from about 2 minutes to about 4 hours, such as about 5 minutes to about 1 hour. The vapor-phase residence time for the digestor may vary from about 1 minute to about 2 hours, for example, such as about 3 minutes to about 30 minutes. The solid-phase, liquid-phase, and vapor-phase residence times may all be about the same, or they may be independently controlled according to reactor-engineering principles (e.g., recycling and internal recirculation strategies).

The aqueous liquor may contain acidifying compounds, such as (but not limited to) sulfuric acid, sulfurous acid, sulfur dioxide, acetic acid, formic acid, or oxalic acid, or combinations thereof. The dilute acid concentration can range from 0.01% to 10% as necessary to improve solubility of particular minerals, such as potassium, sodium, or silica. Preferably, the acid concentration is selected from about 0.01% to 4%, such as 0.1%, 0.5%, 1%, 1.5%, 2%, 2.5%, 3%, or 3.5%.

A second step may include depressurization of the extracted chips into a blow tank or other tank or unit. The vapor can be used for heating the incoming woodchips or cooking liquor, directly or indirectly. The volatilized organic acids (e.g., acetic acid), which are generated or included in the cooking step, may be recycled back to the cooking.

A third step may include mechanically refining the extracted chips. This step (using, for example, a blow-line refiner) may be done before or after depressurization. Optionally, refined solids may be washed. The washing may be accomplished with water, recycled condensates, recycled permeate, or combination thereof. Washing typically removes most of the dissolved material, including hemicelluloses and minerals. The final consistency of the dewatered cellulose-rich solids may be increased to 30% or more, preferably to 50% or more, using a mechanical pressing device. The mechanical pressing device may be integrated with the mechanical refiner, to accomplish combined refining and washing.

A fourth step may include hydrolyzing the extracted chips with enzymes to convert some of the cellulose to glucose. When enzymes are employed for the cellulose hydrolysis, the enzymes preferably include cellulase enzymes. Enzymes may be introduced to the extracted chips along with water, recycled condensates, recycled permeate, additives to adjust pH, additives to enhance hydrolysis (such as lignosulfonates), or combinations thereof.

Some or all of the enzymes may be added to the blow line before or at the blow-line refiner, for example, to assist in enzyme contact with fibers. In some embodiments, at least a portion of enzymes are recycled in a batch or continuous process.

When an acid is employed for the cellulose hydrolysis, the acid may be selected from sulfuric acid, sulfurous acid, sulfur dioxide, formic acid, acetic acid, oxalic acid, or combinations thereof. Acids may be added to the extracted chips before or after mechanical refining. In some embodiments, dilute acidic conditions are used at temperatures between about 100° C. and 190° C., for example about 120° C., 130° C., 140° C., 150° C., 160° C., or 170° C., and preferably from 120° C. to 150° C. In some embodiments, at least a portion of the acid is recycled in a batch or continuous process.

The acid may be selected from sulfuric acid, sulfurous acid, or sulfur dioxide. Alternatively, or additionally, the acid may include formic acid, acetic acid, or oxalic acid from the cooking liquor or recycled from previous hydrolysis.

A fifth step may include conditioning of hydrolysate to remove some or most of the volatile acids and other fermentation inhibitors. The evaporation may include flashing or stripping to remove sulfur dioxide, if present, prior to removal of volatile acids. The evaporation step is preferably performed below the acetic acid dissociation pH of 4.8, and most preferably a pH selected from about 1 to about 2.5. In some embodiments, additional evaporation steps may be employed. These additional evaporation steps may be conducted at different conditions (e.g., temperature, pressure, and pH) relative to the first evaporation step.

In some embodiments, some or all of the organic acids evaporated may be recycled, as vapor or condensate, to the first step (cooking step) to assist in the removal of hemicelluloses or minerals from the biomass. This recycle of organic acids, such as acetic acid, may be optimized along with process conditions that may vary depending on the amount recycled, to improve the cooking effectiveness.

A sixth step may include recovering fermentable sugars, which may be stored, transported, or processed. A sixth step may include fermenting the fermentable sugars to a product, as further discussed below.

A seventh step may include preparing the solid residuals (containing lignin) for combustion. This step may include refining, milling, fluidizing, compacting, and/or pelletizing the dried, extracted biomass. The solid residuals may be fed to a boiler in the form of fine powder, loose fiber, pellets, briquettes, extrudates, or any other suitable form. Using known equipment, solid residuals may be extruded through a pressurized chamber to form uniformly sized pellets or briquettes.

Some embodiments of the invention enable processing of “agricultural residues,” which for present purposes is meant to include lignocellulosic biomass associated with food crops, annual grasses, energy crops, or other annually renewable feedstocks. Exemplary agricultural residues include, but are not limited to, corn stover, corn fiber, wheat straw, sugarcane bagasse, rice straw, oat straw, barley straw, miscanthus, energy cane, or combinations thereof. In certain embodiments, the agricultural residue is sugarcane bagasse.

In some embodiments, the fermentable sugars are recovered from solution, in purified form. In some embodiments, the fermentable sugars are fermented to produce of biochemicals or biofuels such as (but by no means limited to) ethanol, 1-butanol, isobutanol, acetic acid, lactic acid, or any other fermentation products. A purified fermentation product may be produced by distilling the fermentation product, which will also generate a distillation bottoms stream containing residual solids. A bottoms evaporation stage may be used, to produce residual solids.

Following fermentation, residual solids (such as distillation bottoms) may be recovered, or burned in solid or slurry form, or recycled to be combined into the biomass pellets. Use of the fermentation residual solids may require further removal of minerals. Generally, any leftover solids may be used for burning, after concentration of the distillation bottoms.

Alternatively, or additionally, the process may include recovering the residual solids as a fermentation co-product in solid, liquid, or slurry form. The fermentation co-product may be used as a fertilizer or fertilizer component, since it will typically be rich in potassium, nitrogen, and/or phosphorous.

In certain embodiments, the process further comprises combining, at a pH of about 4.8 to 10 or higher, a portion of vaporized acetic acid with an alkali oxide, alkali hydroxide, alkali carbonate, and/or alkali bicarbonate, wherein the alkali is selected from the group consisting of potassium, sodium, magnesium, calcium, and combinations thereof, to convert the portion of the vaporized acetic acid to an alkaline acetate. The alkaline acetate may be recovered. If desired, purified acetic acid may be generated from the alkaline acetate.

The biomass-derived hydrolysate may be the product of acidic or enzymatic hydrolysis, or it may be the extracted solution from the digestor, for example. In some embodiments, the fermentation inhibitor is selected from the group consisting of acetic acid, formic acid, formaldehyde, acetaldehyde, lactic acid, furfural, 5-hydroxymethylfurfural, furans, uronic acids, phenolic compounds, sulfur-containing compounds, and combinations or derivatives thereof.

In certain embodiments, the fermentation inhibitor is acetic acid. The stripped liquid stream preferably has less than 10 g/L acetic acid concentration, such as less than 5 g/L acetic acid concentration.

In some embodiments, the water-rich liquid stream contains biomass solids that are concentrated in the evaporator. These biomass solids may be derived from the same biomass feedstock as is the biomass-derived liquid hydrolysate, in an integrated process.

Optionally, the fermentation inhibitor is recycled to a previous unit operation (e.g., digestor or reactor) for generating the biomass-derived liquid hydrolysate stream, to assist with hydrolysis or pretreatment of a biomass feedstock or component thereof. For example, acetic acid may be recycled for this purpose, to aid in removal of hemicelluloses from biomass and/or in oligomer hydrolysis to monomer sugars.

In some embodiments, the evaporator is a boiler, the water-rich liquid stream comprises boiler feed water, and the evaporated liquid stream comprises boiler condensate.

The process may be continuous, semi-continuous, or batch. When continuous or semi-continuous, the stripping column may be operated countercurrently, cocurrently, or a combination thereof.

The fermentation inhibitor may be selected from the group consisting of acetic acid, formic acid, formaldehyde, acetaldehyde, lactic acid, furfural, 5-hydroxymethylfurfural, furans, uronic acids, phenolic compounds, sulfur-containing compounds, and combinations or derivatives thereof. In some embodiments, the fermentation inhibitor comprises or consists essentially of acetic acid.

In the case of acetic acid, the stripped liquid stream preferably has less than 10 g/L acetic acid concentration, such as less than 5 g/L acetic acid concentration. The rectification column vapor stream preferably has less than 0.5 g/L acetic acid concentration, such as less than 0.1 g/L acetic acid concentration. The rectified liquid stream preferably has at least 25 g/L acetic acid concentration, such as about 40 g/L or more acetic acid. In some embodiments, the rectified liquid stream has at least 10 times higher concentration of acetic acid compared to the stripped liquid stream. In certain embodiments, the process further comprises recovering the acetic acid contained in the rectified liquid stream using liquid-vapor extraction or liquid-liquid extraction.

In some embodiments, the water-rich liquid stream includes evaporator condensate. The evaporator condensate may be derived from an evaporator in which biomass solids are concentrated, and the biomass solids may be derived from the same biomass feedstock as the biomass-derived liquid hydrolysate, in an integrated process.

Optionally, the fermentation inhibitor (e.g., acetic acid) is recycled to a previous unit operation for generating the biomass-derived liquid hydrolysate stream, to assist with hydrolysis or pretreatment of a biomass feedstock or component thereof.

The process may be continuous, semi-continuous, or batch. When continuous or semi-continuous, the stripping column may be operated countercurrently, cocurrently, or a combination thereof. The rectification column may be operated continuous, semi-continuous, or batch.

In various embodiments, the process comprises compressing and/or conveying the rectification column vapor stream using a device selected from the group consisting of a mechanical centrifugal vapor compressor, a mechanical axial vapor compressor, a thermocompressor, an ejector, a diffusion pump, a turbomolecular pump, and combinations thereof.

If desired, a base or other additive may be included in the water-rich liquid stream, or separately introduced to the rectification column, to produce salts or other reaction products derived from fermentation inhibitors. In some embodiments, the water-rich liquid stream includes one or more additives capable of reacting with the fermentation inhibitor. In certain embodiments, the fermentation inhibitor includes acetic acid, and the one or more additives include a base. An acetate salt may then be generated within the rectification column, or in a unit coupled to the rectification column. Optionally, the acetate salt may be separated and recovered using liquid-vapor extraction or liquid-liquid extraction.

Other variations of the invention utilize biomass fractionation with an acid catalyst, a solvent for lignin, and water. This process can fractionate any lignocellulosic-based biomass into its primary major components (cellulose, lignin, and if present, hemicellulose) so that each can be used in potentially distinct processes. An advantage of the process is that it produces cellulose-rich solids while concurrently producing a liquid phase containing a high yield of both hemicellulose sugars and lignin, and low quantities of lignin and hemicellulose degradation products. The flexible fractionation technique enables multiple uses for the products. The cellulose is highly reactive to cellulase enzymes for the manufacture of glucose. Other uses for celluloses can be adjusted based on market conditions.

The biomass feedstock may be selected from hardwoods, softwoods, forest residues, industrial wastes, pulp and paper wastes, consumer wastes, or combinations thereof. Some embodiments utilize agricultural residues, which include lignocellulosic biomass associated with food crops, annual grasses, energy crops, or other annually renewable feedstocks. Exemplary agricultural residues include, but are not limited to, corn stover, corn fiber, wheat straw, sugarcane bagasse, sugarcane straw, rice straw, oat straw, barley straw, miscanthus, energy cane straw/residue, or combinations thereof.

As used herein, “lignocellulosic biomass” means any material containing cellulose and lignin. Lignocellulosic biomass may also contain hemicellulose. Mixtures of one or more types of biomass can be used. In some embodiments, the biomass feedstock comprises both a lignocellulosic component (such as one described above) in addition to a sucrose-containing component (e.g., sugarcane or energy cane) and/or a starch component (e.g., corn, wheat, rice, etc.).

Various moisture levels may be associated with the starting biomass. The biomass feedstock need not be, but may be, relatively dry. In general, the biomass is in the form of a particulate or chip, but particle size is not critical in this invention.

In some embodiments, a first process step is “cooking” (equivalently, “digesting”) which fractionates the three lignocellulosic material components (cellulose, hemicellulose, and lignin) to allow easy downstream removal. Specifically, hemicelluloses are dissolved and over 50% are completely hydrolyzed; cellulose is separated but remains resistant to hydrolysis; and part of the lignin is sulfonated into water-soluble lignosulfonates.

The lignocellulosic material is processed in a solution (cooking liquor) of aliphatic alcohol, water, and sulfur dioxide. The cooking liquor preferably contains at least 10 wt %, such as at least 20 wt %, 30 wt %, 40 wt %, or 50 wt % of a solvent for lignin. For example, the cooking liquor may contain about 30-70 wt % solvent, such as about 50 wt % solvent. The solvent for lignin may be an aliphatic alcohol, such as methanol, ethanol, 1-propanol, 2-propanol, 1-butanol, 2-butanol, isobutanol, 1-pentanol, 1-hexanol, or cyclohexanol. The solvent for lignin may be an aromatic alcohol, such as phenol or cresol. Other lignin solvents are possible, such as (but not limited to) glycerol, methyl ethyl ketone, or diethyl ether. Combinations of more than one solvent may be employed.

Preferably, enough solvent is included in the extractant mixture to dissolve the lignin present in the starting material. The solvent for lignin may be completely miscible, partially miscible, or immiscible with water, so that there may be more than one liquid phase. Potential process advantages arise when the solvent is miscible with water, and also when the solvent is immiscible with water. When the solvent is water-miscible, a single liquid phase forms, so mass transfer of lignin and hemicellulose extraction is enhanced, and the downstream process must only deal with one liquid stream. When the solvent is immiscible in water, the extractant mixture readily separates to form liquid phases, so a distinct separation step can be avoided or simplified. This can be advantageous if one liquid phase contains most of the lignin and the other contains most of the hemicellulose sugars, as this facilitates recovering the lignin from the hemicellulose sugars.

The cooking liquor preferably contains sulfur dioxide and/or sulfurous acid (H₂SO₃). The cooking liquor preferably contains SO₂, in dissolved or reacted form, in a concentration of at least 3 wt %, preferably at least 6 wt %, more preferably at least 8 wt %, such as about 9 wt %, 10 wt %, 11 wt %, 12 wt %, 13 wt %, 14 wt %, 15 wt %, 20 wt %, 25 wt %, 30 wt % or higher. The cooking liquor may also contain one or more species, separately from SO₂, to adjust the pH. The pH of the cooking liquor is typically about 4 or less.

Sulfur dioxide is a preferred acid catalyst, because it can be recovered easily from solution after hydrolysis. The majority of the SO₂ from the hydrolysate may be stripped and recycled back to the reactor. Recovery and recycling translates to less lime required compared to neutralization of comparable sulfuric acid, less solids to dispose of, and less separation equipment. The increased efficiency owing to the inherent properties of sulfur dioxide mean that less total acid or other catalysts may be required. This has cost advantages, since sulfuric acid can be expensive. Additionally, and quite significantly, less acid usage also will translate into lower costs for a base (e.g., lime) to increase the pH following hydrolysis, for downstream operations. Furthermore, less acid and less base will also mean substantially less generation of waste salts (e.g., gypsum) that may otherwise require disposal.

In some embodiments, an additive may be included in amounts of about 0.1 wt % to 10 wt % or more to increase cellulose viscosity. Exemplary additives include ammonia, ammonia hydroxide, urea, anthraquinone, magnesium oxide, magnesium hydroxide, sodium hydroxide, and their derivatives.

The cooking is performed in one or more stages using batch or continuous digestors. Solid and liquid may flow cocurrently or countercurrently, or in any other flow pattern that achieves the desired fractionation. The cooking reactor may be internally agitated, if desired.

Depending on the lignocellulosic material to be processed, the cooking conditions are varied, with temperatures from about 65° C. to 175° C., for example 75° C., 85° C., 95° C., 105° C., 115° C., 125° C., 130° C., 135° C., 140° C., 145° C., 150° C., 155° C., 165° C. or 170° C., and corresponding pressures from about 1 atmosphere to about 15 atmospheres in the liquid or vapor phase. The cooking time of one or more stages may be selected from about 15 minutes to about 720 minutes, such as about 30, 45, 60, 90, 120, 140, 160, 180, 250, 300, 360, 450, 550, 600, or 700 minutes. Generally, there is an inverse relationship between the temperature used during the digestion step and the time needed to obtain good fractionation of the biomass into its constituent parts.

The cooking liquor to lignocellulosic material ratio may be selected from about 1 to about 10, such as about 2, 3, 4, 5, or 6. In some embodiments, biomass is digested in a pressurized vessel with low liquor volume (low ratio of cooking liquor to lignocellulosic material), so that the cooking space is filled with ethanol and sulfur dioxide vapor in equilibrium with moisture. The cooked biomass is washed in alcohol-rich solution to recover lignin and dissolved hemicelluloses, while the remaining pulp is further processed. In some embodiments, the process of fractionating lignocellulosic material comprises vapor-phase cooking of lignocellulosic material with aliphatic alcohol (or other solvent for lignin), water, and sulfur dioxide. See, for example, U.S. Pat. Nos. 8,038,842 and 8,268,125 which are incorporated by reference herein.

A portion or all of the sulfur dioxide may be present as sulfurous acid in the extract liquor. In certain embodiments, sulfur dioxide is generated in situ by introducing sulfurous acid, sulfite ions, bisulfite ions, combinations thereof, or a salt of any of the foregoing. Excess sulfur dioxide, following hydrolysis, may be recovered and reused. In some embodiments, sulfur dioxide is saturated in water (or aqueous solution, optionally with an alcohol) at a first temperature, and the hydrolysis is then carried out at a second, generally higher, temperature. In some embodiments, sulfur dioxide is sub-saturated. In some embodiments, sulfur dioxide is super-saturated. In some embodiments, sulfur dioxide concentration is selected to achieve a certain degree of lignin sulfonation, such as 1%, 2%, 3%, 4%, 5%, 6%, 7%, 8%, 9%, or 10% sulfur content. SO₂ reacts chemically with lignin to form stable lignosulfonic acids which may be present both in the solid and liquid phases.

The concentration of sulfur dioxide, additives, and aliphatic alcohol (or other solvent) in the solution and the time of cook may be varied to control the yield of cellulose and hemicellulose in the pulp. The concentration of sulfur dioxide and the time of cook may be varied to control the yield of lignin versus lignosulfonates in the hydrolysate. In some embodiments, the concentration of sulfur dioxide, temperature, and the time of cook may be varied to control the yield of fermentable sugars.

Once the desired amount of fractionation of both hemicellulose and lignin from the solid phase is achieved, the liquid and solid phases are separated. Conditions for the separation may be selected to minimize the reprecipitation of the extracted lignin on the solid phase. This is favored by conducting separation or washing at a temperature of at least the glass-transition temperature of lignin (about 120° C.).

The physical separation can be accomplished either by transferring the entire mixture to a device that can carry out the separation and washing, or by removing only one of the phases from the reactor while keeping the other phase in place. The solid phase can be physically retained by appropriately sized screens through which liquid can pass. The solid is retained on the screens and can be kept there for successive solid-wash cycles. Alternately, the liquid may be retained and solid phase forced out of the reaction zone, with centrifugal or other forces that can effectively transfer the solids out of the slurry. In a continuous system, countercurrent flow of solids and liquid can accomplish the physical separation.

The recovered solids normally will contain a quantity of lignin and sugars, some of which can be removed easily by washing. The washing-liquid composition can be the same as or different than the liquor composition used during fractionation. Multiple washes may be performed to increase effectiveness. Preferably, one or more washes are performed with a composition including a solvent for lignin, to remove additional lignin from the solids, followed by one or more washes with water to displace residual solvent and sugars from the solids. Recycle streams, such as from solvent-recovery operations, may be used to wash the solids.

After separation and washing as described, a solid phase and at least one liquid phase are obtained. The solid phase contains substantially undigested cellulose. A single liquid phase is usually obtained when the solvent and the water are miscible in the relative proportions that are present. In that case, the liquid phase contains, in dissolved form, most of the lignin originally in the starting lignocellulosic material, as well as soluble monomeric and oligomeric sugars formed in the hydrolysis of any hemicellulose that may have been present. Multiple liquid phases tend to form when the solvent and water are wholly or partially immiscible. The lignin tends to be contained in the liquid phase that contains most of the solvent. Hemicellulose hydrolysis products tend to be present in the liquid phase that contains most of the water.

In some embodiments, hydrolysate from the cooking step is subjected to pressure reduction. Pressure reduction may be done at the end of a cook in a batch digestor, or in an external flash tank after extraction from a continuous digestor, for example. The flash vapor from the pressure reduction may be collected into a cooking liquor make-up vessel. The flash vapor contains substantially all the unreacted sulfur dioxide which may be directly dissolved into new cooking liquor. The cellulose is then removed to be washed and further treated as desired.

A process washing step recovers the hydrolysate from the cellulose. The washed cellulose is pulp that may be used for various purposes (e.g., paper or nanocellulose production). The weak hydrolysate from the washer continues to the final reaction step; in a continuous digestor this weak hydrolysate may be combined with the extracted hydrolysate from the external flash tank. In some embodiments, washing and/or separation of hydrolysate and cellulose-rich solids is conducted at a temperature of at least about 100° C., 110° C., or 120° C. The washed cellulose may also be used for glucose production via cellulose hydrolysis with enzymes or acids.

In another reaction step, the hydrolysate may be further treated in one or multiple steps to hydrolyze the oligomers into monomers. This step may be conducted before, during, or after the removal of solvent and sulfur dioxide. The solution may or may not contain residual solvent (e.g. alcohol). In some embodiments, sulfur dioxide is added or allowed to pass through to this step, to assist hydrolysis. In these or other embodiments, an acid such as sulfurous acid or sulfuric acid is introduced to assist with hydrolysis. In some embodiments, the hydrolysate is autohydrolyzed by heating under pressure. In some embodiments, no additional acid is introduced, but lignosulfonic acids produced during the initial cooking are effective to catalyze hydrolysis of hemicellulose oligomers to monomers. In various embodiments, this step utilizes sulfur dioxide, sulfurous acid, sulfuric acid at a concentration of about 0.01 wt % to 30 wt %, such as about 0.05 wt %, 0.1 wt %, 0.2 wt %, 0.5 wt %, 1 wt %, 2 wt %, 5 wt %, 10 wt %, or 20 wt %. This step may be carried out at a temperature from about 100° C. to 220° C., such as about 110° C., 120° C., 130° C., 140° C., 150° C., 160° C., 170° C., 180° C., 190° C., 200° C., or 210° C. Heating may be direct or indirect to reach the selected temperature.

The reaction step produces fermentable sugars which can then be concentrated by evaporation to a fermentation feedstock. Concentration by evaporation may be accomplished before, during, or after the treatment to hydrolyze oligomers. The final reaction step may optionally be followed by steam stripping of the resulting hydrolysate to remove and recover sulfur dioxide and alcohol, and for removal of potential fermentation-inhibiting side products. The evaporation process may be under vacuum or pressure, from about −0.1 atmospheres to about 10 atmospheres, such as about 0.1 atm, 0.3 atm, 0.5 atm, 1.0 atm, 1.5 atm, 2 atm, 4 atm, 6 atm, or 8 atm.

Recovering and recycling the sulfur dioxide may utilize separations such as, but not limited to, vapor-liquid disengagement (e.g. flashing), steam stripping, extraction, or combinations or multiple stages thereof. Various recycle ratios may be practiced, such as about 0.1, 0.2, 0.3, 0.4, 0.5, 0.6, 0.7, 0.8, 0.9, 0.95, or more. In some embodiments, about 90-99% of initially charged SO₂ is readily recovered by distillation from the liquid phase, with the remaining 1-10% (e.g., about 3-5%) of the SO₂ primarily bound to dissolved lignin in the form of lignosulfonates.

In a preferred embodiment, the evaporation step utilizes an integrated alcohol stripper and evaporator. Evaporated vapor streams may be segregated so as to have different concentrations of organic compounds in different streams. Evaporator condensate streams may be segregated so as to have different concentrations of organic compounds in different streams. Alcohol may be recovered from the evaporation process by condensing the exhaust vapor and returning to the cooking liquor make-up vessel in the cooking step. Clean condensate from the evaporation process may be used in the washing step.

In some embodiments, an integrated alcohol stripper and evaporator system is employed, wherein aliphatic alcohol is removed by vapor stripping, the resulting stripper product stream is concentrated by evaporating water from the stream, and evaporated vapor is compressed using vapor compression and is reused to provide thermal energy.

The hydrolysate from the evaporation and final reaction step contains mainly fermentable sugars but may also contain lignin depending on the location of lignin separation in the overall process configuration. The hydrolysate may be concentrated to a concentration of about 5 wt % to about 60 wt % solids, such as about 10 wt %, 15 wt %, 20 wt %, 25 wt %, 30 wt %, 35 wt %, 40 wt %, 45 wt %, 50 wt % or 55 wt % solids. The hydrolysate contains fermentable sugars.

Fermentable sugars are defined as hydrolysis products of cellulose, galactoglucomannan, glucomannan, arabinoglucuronoxylans, arabinogalactan, and glucuronoxylans into their respective short-chained oligomers and monomer products, i.e., glucose, mannose, galactose, xylose, and arabinose. The fermentable sugars may be recovered in purified form, as a sugar slurry or dry sugar solids, for example. Any known technique may be employed to recover a slurry of sugars or to dry the solution to produce dry sugar solids.

In some embodiments, the fermentable sugars are fermented to produce biochemicals or biofuels such as (but by no means limited to) ethanol, isopropanol, acetone, 1-butanol, isobutanol, lactic acid, succinic acid, or any other fermentation products. Some amount of the fermentation product may be a microorganism or enzymes, which may be recovered if desired.

When the fermentation will employ bacteria, such as Clostridia bacteria, it is preferable to further process and condition the hydrolysate to raise pH and remove residual SO₂ and other fermentation inhibitors. The residual SO₂ (i.e., following removal of most of it by stripping) may be catalytically oxidized to convert residual sulfite ions to sulfate ions by oxidation. This oxidation may be accomplished by adding an oxidation catalyst, such as FeSO4.7H₂O, that oxidizes sulfite ions to sulfate ions, which is a well-known practice for fermentation to acetone/butanol/ethanol (ABE). Preferably, the residual SO₂ is reduced to less than about 100 ppm, 50 ppm, 25 ppm, 10 ppm, 5 ppm, or 1 ppm.

In some embodiments, the process further comprises recovering the lignin as a co-product. The sulfonated lignin may also be recovered as a co-product. In certain embodiments, the process further comprises combusting or gasifying the sulfonated lignin, recovering sulfur contained in the sulfonated lignin in a gas stream comprising reclaimed sulfur dioxide, and then recycling the reclaimed sulfur dioxide for reuse.

The process lignin separation step is for the separation of lignin from the hydrolysate and can be located before or after the final reaction step and evaporation. If located after, then lignin will precipitate from the hydrolysate since alcohol has been removed in the evaporation step. The remaining water-soluble lignosulfonates may be precipitated by converting the hydrolysate to an alkaline condition (pH higher than 7) using, for example, an alkaline earth oxide, preferably calcium oxide (lime). The combined lignin and lignosulfonate precipitate may be filtered. The lignin and lignosulfonate filter cake may be dried as a co-product or burned or gasified for energy production. The hydrolysate from filtering may be recovered and sold as a concentrated sugar solution product or further processed in a subsequent fermentation or other reaction step.

Native (non-sulfonated) lignin is hydrophobic, while lignosulfonates are hydrophilic. Hydrophilic lignosulfonates may have less propensity to clump, agglomerate, and stick to surfaces. Even lignosulfonates that do undergo some condensation and increase of molecular weight, will still have an HSO₃ group that will contribute some solubility (hydrophilic).

In some embodiments, the soluble lignin precipitates from the hydrolysate after solvent has been removed in the evaporation step. In some embodiments, reactive lignosulfonates are selectively precipitated from hydrolysate using excess lime (or other base, such as ammonia) in the presence of aliphatic alcohol. In some embodiments, hydrated lime is used to precipitate lignosulfonates. In some embodiments, part of the lignin is precipitated in reactive form and the remaining lignin is sulfonated in water-soluble form.

The process fermentation and distillation steps are intended for the production of fermentation products, such as alcohols or organic acids. After removal of cooking chemicals and lignin, and further treatment (oligomer hydrolysis), the hydrolysate contains mainly fermentable sugars in water solution from which any fermentation inhibitors have been preferably removed or neutralized. The hydrolysate is fermented to produce dilute alcohol or organic acids, from 1 wt % to 20 wt % concentration. The dilute product is distilled or otherwise purified as is known in the art.

When alcohol is produced, such as ethanol, some of it may be used for cooking liquor makeup in the process cooking step. Also, in some embodiments, a distillation column stream, such as the bottoms, with or without evaporator condensate, may be reused to wash cellulose. In some embodiments, lime may be used to dehydrate product alcohol. Side products may be removed and recovered from the hydrolysate. These side products may be isolated by processing the vent from the final reaction step and/or the condensate from the evaporation step. Side products include furfural, hydroxymethyl furfural (HMF), methanol, acetic acid, and lignin-derived compounds, for example.

The cellulose-rich material is highly reactive in the presence of industrial cellulase enzymes that efficiently break the cellulose down to glucose monomers. It has been found experimentally that the cellulose-rich material, which generally speaking is highly delignified, rapidly hydrolyzes to glucose with relatively low quantities of enzymes. For example, the cellulose-rich solids may be converted to glucose with at least 80% yield within 24 hours at 50° C. and 2 wt % solids, in the presence of a cellulase enzyme mixture in an amount of no more than 15 filter paper units (FPU) per g of the solids. In some embodiments, this same conversion requires no more than 5 FPU per g of the solids.

The glucose may be fermented to an alcohol, an organic acid, or another fermentation product. The glucose may be used as a sweetener or isomerized to enrich its fructose content. The glucose may be used to produce baker's yeast. The glucose may be catalytically or thermally converted to various organic acids and other materials.

In some embodiments, the cellulose-rich material is further processed into one more cellulose products. Cellulose products include market pulp, dissolving pulp (also known as α-cellulose), fluff pulp, purified cellulose, paper, paper products, and so on. Further processing may include bleaching, if desired. Further processing may include modification of fiber length or particle size, such as when producing nanocellulose or nanofibrillated or microfibrillated cellulose. It is believed that the cellulose produced by this process is highly amenable to derivatization chemistry for cellulose derivatives and cellulose-based materials such as polymers.

When hemicellulose is present in the starting biomass, all or a portion of the liquid phase contains hemicellulose sugars and soluble oligomers. It is preferred to remove most of the lignin from the liquid, as described above, to produce a fermentation broth which will contain water, possibly some of the solvent for lignin, hemicellulose sugars, and various minor components from the digestion process. This fermentation broth can be used directly, combined with one or more other fermentation streams, or further treated. Further treatment can include sugar concentration by evaporation; addition of glucose or other sugars (optionally as obtained from cellulose saccharification); addition of various nutrients such as salts, vitamins, or trace elements; pH adjustment; and removal of fermentation inhibitors such as acetic acid and phenolic compounds. The choice of conditioning steps should be specific to the target product(s) and microorganism(s) employed.

In some embodiments, hemicellulose sugars are not fermented but rather are recovered and purified, stored, sold, or converted to a specialty product. Xylose, for example, can be converted into xylitol.

A lignin product can be readily obtained from a liquid phase using one or more of several methods. One simple technique is to evaporate off all liquid, resulting in a solid lignin-rich residue. This technique would be especially advantageous if the solvent for lignin is water-immiscible. Another method is to cause the lignin to precipitate out of solution. Some of the ways to precipitate the lignin include (1) removing the solvent for lignin from the liquid phase, but not the water, such as by selectively evaporating the solvent from the liquid phase until the lignin is no longer soluble; (2) diluting the liquid phase with water until the lignin is no longer soluble; and (3) adjusting the temperature and/or pH of the liquid phase. Methods such as centrifugation can then be utilized to capture the lignin. Yet another technique for removing the lignin is continuous liquid-liquid extraction to selectively remove the lignin from the liquid phase, followed by removal of the extraction solvent to recover relatively pure lignin.

Lignin produced in accordance with the invention can be used as a fuel. As a solid fuel, lignin is similar in energy content to coal. Lignin can act as an oxygenated component in liquid fuels, to enhance octane while meeting standards as a renewable fuel. The lignin produced herein can also be used as polymeric material, and as a chemical precursor for producing lignin derivatives. The sulfonated lignin may be sold as a lignosulfonate product, or burned for fuel value.

The present invention also provides systems configured for carrying out the disclosed processes, and compositions produced therefrom. Any stream generated by the disclosed processes may be partially or completed recovered, purified or further treated, and/or marketed or sold.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. An apparatus for enzymatically hydrolyzing pretreated lignocellulosic biomass, said apparatus including at least one clarifying vessel comprising:
(a) an inlet well, disposed with walls at or near the top of said clarifying vessel, configured for continuously feeding pretreated lignocellulosic biomass;
(b) one or more overflow weirs configured to allow liquid-phase hydrolysate to be continuously or periodically recovered from said clarifying vessel; and
(c) a controllable bottom outlet to allow a solid phase or solid slurry to be continuously or periodically recovered from said clarifying vessel, separately from said liquid-phase hydrolysate.

2. The apparatus of claim 1, wherein said walls of said well extend down below the local elevation of said weirs.

3. The apparatus of claim 1, wherein said controllable bottom outlet is further configured with a rake for continuously or periodically assisting in removal of said solid phase or solid slurry.

4. The apparatus of claim 1, wherein said clarifying vessel is capable of operating at a solids concentration of at least 10 wt %.

5. The apparatus of claim 4, wherein said solids concentration is at least 20 wt %.

6. The apparatus of claim 1, said apparatus further comprising a recycle line for recycling at least a portion of said hydrolysate back to said inlet well.

7. The apparatus of claim 1, said apparatus including at least two clarifying vessels in series.

8. The apparatus of claim 1, said apparatus including at least two clarifying vessels in parallel.

9. The apparatus of claim 1, wherein said clarifying vessel is an existing recausticizing clarifiers in a pulp and paper mill.

10. The apparatus of claim 1, said apparatus including at least two clarifying vessels in series or in parallel, wherein each of said clarifying vessels is an existing recausticizing clarifier in a pulp and paper mill.

11. A method of enzymatically hydrolyzing pretreated lignocellulosic biomass, said method comprising:
(a) providing pretreated lignocellulosic biomass containing cellulose and lignin;
(b) introducing said pretreated lignocellulosic biomass to a clarifying vessel under effective hydrolysis conditions and in the presence of enzymes including cellulases, to hydrolyze said cellulose to a liquid-phase hydrolysate;
(c) separating, by sedimentation within said clarifying vessel, said hydrolysate from said lignin; and
(d) recovering said hydrolysate from one or more overflow weirs of said clarifying vessel.

12. The method of claim 11, wherein said pretreated lignocellulosic biomass is chemically pretreated, physically pretreated, or a combination thereof and is optionally premixed with enzymes.

13. The method of claim 11, wherein said pretreated lignocellulosic biomass is introduced into a well, disposed with walls, at or near the top of said clarifying vessel.

14. The method of claim 11, wherein said effective hydrolysis conditions include a hydrolysis residence time selected from about 4 hours to about 72 hours; and/or a hydrolysis pH selected from about 4 to about 7; and/or a hydrolysis temperature selected from about 30° C. to about 70° C.

15. The method of claim 11, wherein said method is continuous or semi-continuous.

16. The method of claim 11, wherein said lignin is recovered continuously or periodically from a bottom stream output of said clarifying vessel.

17. The method of claim 11, said method further comprising introducing a flocculating agent to said clarifying vessel.

18. The method of claim 11, said method further comprising introducing a surfactant to said clarifying vessel.

19. The method of claim 11, wherein said step (b) is conducted at a solids concentration of at least 10 wt %.

20. The method of claim 19, wherein said solids concentration is at least 20 wt %.
